(19) United States
(12) Plant Patent Application Publication
(10) Pub. No.: US 2012/0255077 P1
(43) Pub. Date: Oct. 4, 2012

(54) Bracteantha plant named 'KLEBB10710'

(50) ;

(75) Inventors: Nils Klemm, Stuttgart (DE); Martin Glawe, Stuttgart (DE)

(73) Assignee: KLEMM+SOHN GMBH & CO. KG, Stuttgart (DE)

(21) Appl. No.: 13/065,781

(22) Filed: Mar. 30, 2011

(51) Int. Cl. A01H 5/00

(52) U.S. Cl. PLT/263.1

(57) ABSTRACT

A new cultivar of Bracteantha named ‘KLEBB10710’ particularly characterized by dark pink flowers and good branching, is disclosed.

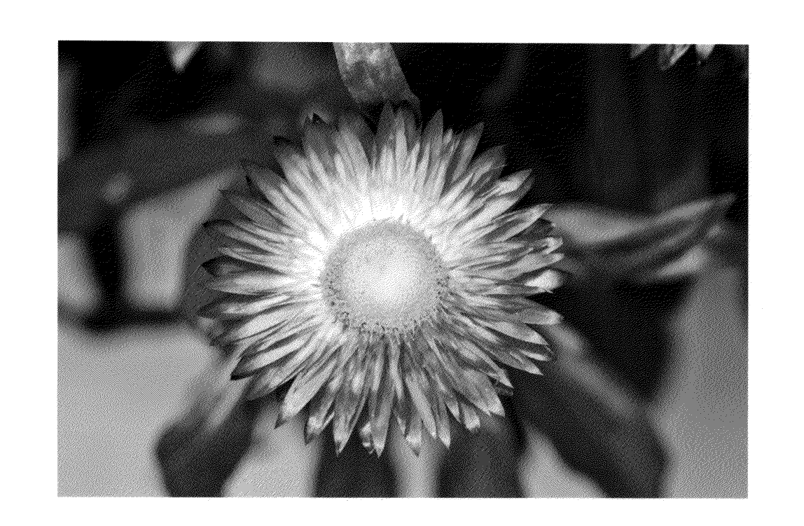

### GENUS AND SPECIES

Bracteantha bracteata

### VARIETY DENOMINATION

‘KLEBB10710’

### BACKGROUND OF THE NEW PLANT

The present invention comprises a new and distinct cultivar of Bracteantha, botanically known as Bracteantha bracteata, and hereinafter referred to by the cultivar name ‘KLEBB10710’. ‘KLEBB10710’ was discovered as a seedling resulting from a cross in July 2006 in Stuttgart, Germany between the proprietary female Bracteantha plant, ‘BB 06 076’ (unpatented), and the proprietary male Bracteantha plant, ‘BB 04 0025’ (unpatented). A single plant selection was subsequently chosen for further evaluation and asexual propagation.

The new variety was first propagated via vegetative cuttings in July 2007 in Stuttgart, Germany and has been asexually reproduced repeatedly by vegetative cuttings in Stuttgart, Germany for approximately seven to eight generations. ‘KLEBB10710’ has been found to retain its distinctive characteristics through successive asexual propagations via vegetative cuttings.

Plant Breeder's Rights for this variety have not been applied for. ‘KLEBB10710’ has not been made publicly available or sold more than one year prior to the filing date of this application.

### SUMMARY OF THE INVENTION

The following are the most outstanding and distinguishing characteristics of this new variety when grown under normal horticultural practices in a glass greenhouse in Stuttgart, Germany.

1. Dark pink flowers; and

2. Good branching.

### DESCRIPTION OF THE PHOTOGRAPH

This new Bracteantha plant is illustrated by the accompanying photograph which shows a close-up of the mature flower. The colors shown are as true as can be reasonably obtained by conventional photographic procedures. The photograph is of a plant about 3 months old taken in the early Spring of 2010 grown from a rooted cutting in a 12 centimeter pot in a glass greenhouse in Stuttgart, Germany under conditions which approximate those generally used in normal horticultural practice.

### DESCRIPTION OF THE NEW CULTIVAR

The following detailed descriptions set forth the distinctive characteristics of ‘KLEBB10710’. The data which define these characteristics were collected from asexual reproductions carried out in Stuttgart, Germany. The plant history was taken in the early Spring of 2010 on 3-month old plants grown in 12 centimeter pots in a glass greenhouse in Stuttgart, Germany. The plants were pinched once 10 days after planting. The color readings were determined under natural light. Color references are primarily to the RHS Colour Chart of The Royal Horticultural Society of London (R.H.S.), Fifth Edition (2007).

### DESCRIPTION OF THE NEW PLANT

Classification:
   Family.—Asteraceae.
   Genus and species.—Bracteantha bracteata.
Parentage:
   Female parent.—The proprietary Bracteantha parent, ‘BB 06 076’ (unpatented).
   Male parent.—The proprietary Bracteantha parent, ‘BB 04 0025’ (unpatented).
Plant:
   Form.—Pot plant, annual.
   Growth habit.—Round, bushy.
   Propagation type.—Vegetative cuttings, pot plant.
   Height from soil level to top of plant plane.—25.0 cm to 30.0 cm.
   Diameter (area of spread).—25.0 cm.
   Time to initiate roots.—15 days.
   Time to produce a rooted cutting.—18 days.
   Root description and habit.—Fine, small, white.
Branches:
   Type.—Lateral branches.
   Quantity per plant.—6 to 7.
   Length.—10.0 cm to 20.0 cm.
   Diameter.—4.0 cm to 6.0 cm.
   Internode length.—0.5 cm to 1.0 cm.
   Aspect.—Upright.
   Strength.—Medium to weak.
   Texture.—Rough.
   Color.13 RHS 137C.
Leaves:
   Arrangement.—Opposite.
   Length.—4.0 cm to 10.0 cm.
   Width.—0.5 cm to 2.0 cm.
   Shape.—Ovate.
   Apex.—Acute.
   Base.—Alternate.
   Margin.—Entire.
   Texture (both upper and lower surface).—Smooth.
   Venation pattern.—Pinnate.
   Venation color (both upper and lower surface).—RHS 137D.
   Color.—Immature leaf (both surfaces): RHS 137D Mature leaf: Upper surface: RHS 137C Lower surface: RHS 137D.
   Petiole.—Length: 2.0 cm to 4.0 cm Diameter: 0.3 cm to 0.6 cm Color: RHS 137C.
Inflorescence:
   Appearance.—Umbel (dichasium).
   Flowering habit and timing.—Continuous from Spring to Fall.
   Quantity of inflorescences on the plant.—5 to 7.
   Fragrance.—Absent.
   Lastingness of inflorescences on the plant.—14 days.
   Diameter.—8.0 cm.
   Depth (height).—3.5 cm.
   Disc diameter.—4.5 cm.
   Receptacle diameter.—3.0 cm.
   Receptacle height.—2.5 cm.
Bud:
   Height.—0.5 cm to 1.2 cm.
   Diameter.—0.5 cm to 0.8 cm.
   Shape.—Elliptic.
   Color.—RHS 59A.
Involucral bracts:
   Shape.—Lanceolate to ovate.
   Length, largest bracts.—1.0 cm.
   Width, largest bracts.—0.5 cm.
   Apex.—Acute.
   Base.—Attenuate.
   Margin.—Entire.
   Texture.—Smooth.
   Color (when opening).—Upper surface: RHS 137C Lower surface: RHS 137D.
   Color (when fully opened).—Upper surface: RHS 137A Lower surface: RHS 137C.
   Number of involucral bracts per inflorescence.—Varies.
Disc florets:
   Arrangement.—Radial.
   Shape.—Lanceolate to ovate.
   Apex.—Acute.
   Length.—2.0 cm.
   Diameter, apex.—0.1 cm.
   Diameter, base.—0.3 cm.
   Color.—Immature: RHS 59B Mature: RHS 61A Apex: RHS 61A Mid-section: RHS 61B Base: RHS 61D.
   Number of disc florets per inflorescence.—Greater than 50.
Phyllaries:
   Quantity per inflorescence.—8 to 10.
   Length.—0.4 cm.
   Width.—0.4 cm.
   Shape.—Elliptic to round.
   Apex.—Acute.
   Base.—Obtuse.
   Margin.—Entire.
   Texture (both the upper and lower surfaces).—Smooth.
   Color (both upper and lower surface).—RHS 165D.
Comparision with parental and commercial cultivars: ‘KLEBB10710’ differs from the female parent, the proprietary Bracteantha plant, ‘BB 06 076’ (unpatented), in that ‘KLEBB10710’ has dark pink flowers and a medium compact growth habit, while ‘BB 06 076’ has light pink flowers and a strong growth habit. ‘KLEBB10710’ differs from the male parent, the proprietary Bracteantha plant, ‘BB 04 0025’ (unpatented), in that ‘KLEBB10710’ has a medium compact growth habit, while ‘BB 04 0025’ has a strong growth habit. In addition, the flowers of ‘KLEBB10710’ are smaller than those of ‘BB 04 0025’. ‘KLEBB10710’ differs from the commercial cultivar, ‘Mohave Dark Pink’ (unpatented), in that ‘KLEBB10710’ has better branching, smaller flowers, and an earlier flowering response than ‘Mohave Dark Pink’.

We claim:

1. A new and distinct cultivar of Bracteantha plant named ‘KLEBB10710’ as shown and described herein.
